(19) United States
(12) Patent Application Publication
(10) Pub. No.: US 2008/0241394 A1
(43) Pub. Date: Oct. 2, 2008

(54) Dyes and their use in ink-jet printing

(75) Inventor: Gavin WRIGHT, Blackley (GB)

Correspondence Address: MORGAN LEWIS & BOCKIUS LLP, 1111 PENNSYLVANIA AVENUE NW, WASHINGTON, DC, 20004, US

(73) Assignee: Fujifilm Imaging Colorants Limited

(21) Appl. No.: 12/078,303

(22) Filed: Mar. 28, 2008

(30) Foreign Application Priority Data
Mar. 30, 2007 (GB) 0706224.3

(51) Int. Cl. B05D 5/04; C07C 245/10; C09D 11/02; G01D 11/00

(52) U.S. Cl. 427/256; 534/780; 534/778; 534/767; 106/31.13; 347/100

(57) ABSTRACT

A compound of Formula (1) or a salt thereof:

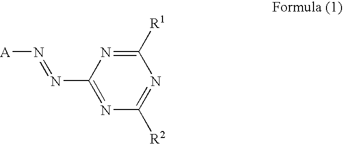

wherein:

A is optionally substituted aryl or optionally substituted heterocyclyl; and

R¹ and R² are independently H or a substituent. Also metal chelates, compositions, inks, printing processes, printed material and ink-jet cartridges.

This invention relates to compounds and their metal chelates, compositions and inks, to printing processes, to printed substrates and to ink-jet printer cartridges.

Ink-jet printing is a non-impact printing technique in which droplets of ink are ejected through a fine nozzle onto a substrate without bringing the nozzle into contact with the substrate. The set of inks used in this technique typically comprise yellow, magenta, cyan and black inks.

With the advent of high-resolution digital cameras and ink-jet printers it is becoming increasingly common for consumers to print photographs using an ink-jet printer. This avoids the expense of conventional silver halide photography and provides a print quickly and conveniently.

While ink-jet printers have many advantages over other forms of printing and image development there are still technical challenges to be addressed. For example, there are the contradictory requirements of providing ink colorants that are soluble in the ink medium and yet do not run or smudge excessively when printed on paper. The inks need to dry quickly to avoid sheets sticking together after they have been printed, but they should not form a crust over the tiny nozzle used in the printer. Storage stability is also important to avoid particle formation that could block the tiny nozzles used in the printer especially since consumers can keep an ink-jet ink cartridge for several months. Furthermore, and especially important with photographic quality reproductions, the resultant images should not bronze or fade rapidly on exposure to light or common oxidising gases such as ozone.

Azo dyes and pigments are by far the most important class of colorants. They are easy to prepare from readily available starting materials, tinctorially strong, cover the whole shade range and have good fastness properties. The key step in forming an azo colorant is the diazo coupling. However, the diazonium component is only a weak electrophile and so the aromatic ring to which it is coupled must be highly activated. Because of this azo colorants have not been prepared from certain ring systems.

According to a first aspect of the present invention there is provided a compound of Formula (1) or a salt thereof:

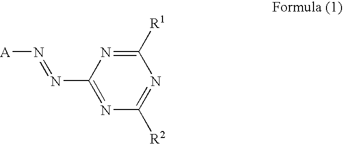

wherein:

A is optionally substituted aryl or optionally substituted heterocyclyl; and

R¹ and R² are independently H or a substituent.

Preferably at least one of A, R¹ or R² carries at least one acidic water solubilising group selected from the group consisting of —SO₃H, —CO₂H, —PO₃H₂ and —COSH.

Preferably A is optionally substituted phenyl, optionally substituted naphthyl, optionally substituted phenanthrenyl or optionally substituted 5- or 6-membered heterocyclyl.

More preferably A comprises a S, N or O either in the ring or as a substituent which is in such a position that it is, along with a N in the azo bond and a N in the triazinyl component is able to ligate to a metal and so form a metal complex comprising a compound of Formula (1).

Preferably A carries at least one acidic water solubilising group selected from the group consisting of —SO₃H, —CO₂H, —PO₃H₂ and —COSH, especially —SO₃H and —CO₂H.

More preferably A is 1-hydroxy-2-naphthyl, and analogues thereof especially analogues carrying at least one acidic water solubilising group selected from the group consisting of —SO₃H, —CO₂H, —PO₃H₂ and —COSH, for example 1-hydroxy-4-sulpho-2-naphthyl; 1-hydroxy-5-sulpho-2-naphthyl and 9-hydroxy-10-phenanthrenyl-2,7-disulfonic acid.

Preferably R¹, R² and the optional substituents on A are independently selected from: optionally substituted alkyl (preferably optionally substituted C₁₋₈-alkyl, especially C₁₋₄-alkyl), optionally substituted alkoxy (preferably optionally substituted C₁₋₈-alkoxy, especially C₁₋₄-alkoxy), optionally substituted aryl (preferably optionally substituted phenyl or optionally substituted naphthyl), optionally substituted aryloxy (preferably phenoxy), optionally substituted heterocyclyl, polyalkylene oxide (preferably polyethylene oxide or polypropylene oxide), nitro, cyano, halo, ureido, —SO₂F, hydroxy, ester, —NR^{a}R^{b}, —COR^{a}, —CONR^{a}R^{b}, —NHCOR^{a}, carboxyester, sulfone, and —SO₂NR^{a}R^{b}, wherein R^{a} and R^{b} are each independently H or optionally substituted alkyl (especially optionally substituted C₁₋₄-alkyl). Optional substituents for any of the substituents described above may be selected from the same list of substituents.

Especially preferred optional substituents on A, other than —SO₃H, —CO₂H, —PO₃H₂ and —COSH, are: —OH; —NR^{a}R^{b}, wherein R^{a} and R^{b} are each independently H or optionally substituted alkyl (especially optionally substituted C₁₋₄-alkyl); CN.

More preferably R¹ and R² are independently selected from; H; —NR^{a}R^{b}, wherein R^{a} and R^{b} are each independently H or optionally substituted alkyl (especially optionally substituted C₁₋₄-alkyl), optionally substituted C₁₋₄-alkoxy (especially methoxy), SH or SR^{c}, wherein R^{c} is optionally substituted alkyl.

Preferably the compounds of Formula (1) are water-soluble, more preferably they have a solubility in water at 20° C. of 0.5 to 15 and more preferably 0.6 to 10% by weight.

The compounds of Formula (1) provide prints which exhibit a high light-fastness, ozone fastness, wet fastness and good optical density.

Compounds of Formula (1) are preferably free from fibre reactive groups. The term fibre reactive group is well known in the art and is described for example in EP 0356014 A1. Fibre reactive groups are capable, under suitable conditions, of reacting with the hydroxyl groups present in cellulosic fibres or with the amino groups present in natural fibres to form a covalent linkage between the fibre and the dye. As examples of fibre reactive groups preferably not present in the compounds of the first aspect of the present invention there may be mentioned aliphatic sulfonyl groups which contain a sulfate ester group in the beta-position to the sulfur atom, e.g. beta-sulfato-ethylsulfonyl groups, alpha, beta-unsaturated acyl radicals of aliphatic carboxylic acids, for example acrylic acid, alpha-chloro-acrylic acid, alpha-bromoacrylic acid, propiolic acid, maleic acid and mono- and dichloro maleic; also the acyl radicals of acids which contain a substituent which reacts with cellulose in the presence of an alkali, e.g. the radical of a halogenated aliphatic acid such as chloroacetic acid, beta-chloro and beta-bromopropionic acids and alpha, beta-dichloro- and dibromopropionic acids or radicals of vinylsulfonyl- or beta-chloroethylsulfonyl- or beta-sulfatoethyl-sulfonyl-endo-methylene cyclohexane carboxylic acids. Other examples of cellulose reactive groups are tetrafluorocyclobutyl carbonyl, trifluoro-cyclobutenyl carbonyl, tetrafluorocyclobutylethenyl carbonyl, trifluoro-cyclobutenylethenyl carbonyl; activated halogenated 1,3-dicyanobenzene radicals; and heterocyclic radicals which contain 1, 2 or 3 nitrogen atoms: in the heterocyclic ring and at least one cellulose reactive substituent on a carbon atom of the ring.

The compounds of Formula (1) may be in the free acid or salt form. Preferred salts are water-soluble, for example alkali metal salts, especially lithium, sodium, potassium, ammonium, substituted ammonium and mixed salts thereof. Preferred alkali metal salts are those with sodium or lithium ammonium and substituted alkyl ammonium salts.

Preferred ammonium and substituted ammonium salts have cations of the formula ⁺NV₄ wherein each V independently is H or optionally substituted alkyl, or two groups represented by V are H or optionally substituted alkyl and the remaining two groups represented by V, together with the N atom to which they are attached, form a 5- or 6-membered ring (preferably a morpholinyl, pyridinyl or piperidinyl ring).

Preferably each V independently is H or C₁₋₄-alkyl, more preferably H, CH₃ or CH₃CH₂, especially H.

Examples of cations include ⁺NH₄, morpholinium, piperidinium, pyridinium, (CH₃)₃N⁺H, (CH₃)₂N⁺H₂, H₂N⁺(CH₃)(CH₂CH₃), CH₃N⁺H₃, CH₃CH₂N⁺H₃, H₂N⁺(CH₂CH₃)₂, CH₃CH₂CH₂N⁺H₃, (CH₃)₂CHN⁺H₃, N⁺(CH₃)₄, N⁺(CH₂CH₃)₄, N-methylpyridinium, N,N-dimethyl piperidinium and N,N-dimethyl morpholinium.

Sodium, lithium, potassium, ammonium, or substituted ammonium salts are preferred because we have found that these salts can provide prints which exhibit a high light-fastness when incorporated into an ink-jet printing ink.

The compounds of Formula (1) may exist in tautomeric forms other than those shown in this specification. These tautomers are included within the scope of the present invention.

The compounds of Formula (1) may be prepared by method known in the art.

Preferably they are prepared by, for example, condensing a compound of Formula (2)

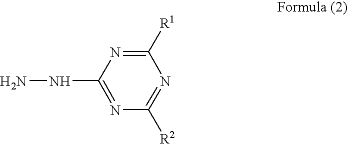

wherein R¹ and R² are as defined above, with an aryl or heteroaryl quinone.

Compounds of Formula (2) may be prepared by known literature methods, for example by reaction of a suitable substituted halo-heterocycle with hydrazine or by reaction of a carbonyl equivalent with phosphorous oxychloride (POCl₃) and then hydrazine.

Many aryl or heteroaryl quinones are commercially available other may be readily prepared by methods well known in the art such as those described in the examples of WO06/125951 which is hereby incorporated by reference.

The product of the above process may be converted to a salt by conventional techniques as hereinbefore described. Alternatively the product may be isolated in its free acid form by acidifying the reaction mixture, preferably using a mineral acid, for example hydrochloric acid, and when the product precipitates as a solid it may be separated from the mixture by filtration. Unwanted anions may be, and preferably are, removed from the product of the process by dialysis, osmosis, ultrafiltration or a combination thereof. Alternatively, the product solution is subjected to the above purification directly without isolation of the product.

A second aspect of the invention provides a metal chelate compound comprising a compound of Formula (1).

The metal in the metal chelate compound comprising a compound of Formula (1) is preferably one or more of: boron, nickel, chromium, cobalt, copper, zinc, iron or manganese.

More preferably the metal in the metal chelate compound comprising a compound of Formula (1) is nickel or copper.

The compound of Formula (1) is preferably chelated to the metal in the ratio 1:1, 2:1, 2:2 or 2:3 respectively, especially in the ratio 1:1 or 2:1 respectively. The metal chelate compounds comprising a compound of Formula (1) may also comprise mixtures of these different chelated forms.

The compounds of Formula (1) in the metal chelate compounds comprising a compound of Formula (1) are as described and preferred in the first aspect of the invention.

When there is more than one ligand of Formula (1) present in the metal chelate compound then the ligands of Formula (1) may be the same or different but preferably they are the same.

The metal-chelate compound comprising a compound of Formula (1) may also comprise 1 or more additional ligands. These ligands may be coloured or colourless and when there is more than one they may be the same or different.

The metal chelate compounds comprising a compound of Formula (1), as described herein, may exist in tautomeric forms other than those shown in this specification. These tautomers are also included within the scope of the present inventions.

The metal chelate compounds comprising a compound of Formula (1) may also exist in different geometries e.g. octahedral or square planar. These different geometric forms are also included in the scope of the present invention.

The metal chelate compounds comprising a compound of Formula (1) may also be present as a mixture with the compounds of Formula (1), either as free acids or as salts, which may be either the same or different from the compound in the metal chelate compound.

Preferably the metal chelate compounds comprising a compounds of Formula (1) are water-soluble, more preferably they have a solubility in water at 20° C. of 0.5 to 15, more preferably 0.6 to 10% by weight.

The metal chelate compounds of the first aspect of the present invention provide prints which exhibit a high light-fastness, ozone fastness, wet fastness and good optical density.

The compounds of Formula (1) and the metal chelate compound comprising a compound of Formula (1) may be in the free acid or salt form. Preferred salts are water-soluble salts as described in the first aspect of the invention.

The metal chelate compounds may be prepared using techniques that are well known in the art. For example a suitable method comprises mixing a solution of a metal salt and a solution of a compound of Formula (1), preferably in aqueous solution.

Normally 0.5 to 24 hours is sufficient for the metal salt to form a metal chelate compound with the compound of Formula (1).

The product of the above process may be converted to a salt by conventional techniques as described in the first aspect of the invention.

The present invention also covers mixtures comprising two or more different metal chelate compounds comprising a compound of Formula (1) of the present invention or salts thereof.

According to a third aspect of the present invention there is provided a composition comprising a compound of Formula (1) as described in the first aspect of the invention or a metal chelate compound comprising a compound of Formula (1) as described in the second aspect of the invention, or a mixture thereof, and a liquid medium.

Preferred compositions according to the second aspect of the invention comprise:
(a) from 0.01 to 30 parts of a compound of Formula (1) according to the first aspect of the invention, a metal chelate compound comprising a compound of Formula (1) as described in the second aspect of the invention or a mixture thereof; and
(b) from 70 to 99.99 parts of a liquid medium;
   wherein all parts are by weight

Preferably the number of parts of (a)+(b)=100.

The number of parts of component (a) is preferably from 0.1 to 20, more preferably from 0.5 to 15, and especially from 1 to 5 parts. The number of parts of component (b) is preferably from 80 to 99.9, more preferably from 85 to 99.5 and especially from 95 to 95 parts.

Preferably component (a) is completely dissolved in component (b). Preferably component (a) has a solubility in component (b) at 20° C. of at least 10%. This allows the preparation of liquid dye concentrates that may be used to prepare more dilute inks and reduces the chance of the dye precipitating if evaporation of the liquid medium occurs during storage.

The inks may be incorporated in an ink-jet printer as a high concentration ink, a low concentration ink or both a high concentration and a low concentration ink. In the latter case this can lead to improvements in the resolution and quality of printed images. Thus the present invention also provides a composition (preferably an ink) where component (a) is present in an amount of 2.5 to 7 parts, more preferably 2.5 to 5 parts (a high concentration ink) or component (a) is present in an amount of 0.5 to 2.4 parts, more preferably 0.5 to 1.5 parts (a low concentration ink).

Preferred liquid media include water, a mixture of water and organic solvent and organic solvent free from water. Preferably the liquid medium comprises a mixture of water and organic solvent or organic solvent free from water.

When the liquid medium (b) comprises a mixture of water and organic solvent, the weight ratio of water to organic solvent is preferably from 99:1 to 1:99, more preferably from 99:1 to 50:50 and especially from 95:5 to 80:20.

It is preferred that the organic solvent present in the mixture of water and organic solvent is a water-miscible organic solvent or a mixture of such solvents. Preferred water-miscible organic solvents include C₁₋₆-alkanols, preferably methanol, ethanol, n-propanol, isopropanol, n-butanol, sec-butanol, tert-butanol, n-pentanol, cyclopentanol and cyclohexanol; linear amides, preferably dimethylformamide or dimethylacetamide; ketones and ketone-alcohols, preferably acetone, methyl ether ketone, cyclohexanone and diacetone alcohol; water-miscible ethers, preferably tetrahydrofuran and dioxane; diols, preferably diols having from 2 to 12 carbon atoms, for example ethylene glycol, propylene glycol, butylene glycol, pentylene glycol, hexylene glycol and thiodiglycol and oligo- and poly-alkyleneglycols, preferably diethylene glycol, triethylene glycol, polyethylene glycol and polypropylene glycol; triols, preferably glycerol and 1,2,6-hexanetriol; mono-C₁₋₄-alkyl ethers of diols, preferably mono-C₁₋₄-alkyl ethers of diols having 2 to 12 carbon atoms, especially 2-methoxyethanol, 2-(2-methoxyethoxy)ethanol, 2-(2-ethoxyethoxy)-ethanol, 2-[2-(2-methoxyethoxy)ethoxy]ethanol, 2-[2-(2-ethoxyethoxy)-ethoxy]-ethanol and ethyleneglycol monoallylether; cyclic amides, preferably 2-pyrrolidone, N-methyl-2-pyrrolidone, N-ethyl-2-pyrrolidone, caprolactam and 1,3-dimethylimidazolidone; cyclic esters, preferably caprolactone; sulfoxides, preferably dimethyl sulfoxide; and sulfones, such as sulfolane.

Preferably the liquid medium comprises water and 2 or more, especially from 2 to 8, water-miscible organic solvents.

Especially preferred water-miscible organic solvents are cyclic amides, especially 2-pyrrolidone, N-methyl-pyrrolidone and N-ethyl-pyrrolidone; diols, especially 1,5-pentane diol, ethyleneglycol, thiodiglycol, diethyleneglycol and triethyleneglycol; and mono-C₁₋₄-alkyl and C₁₋₄-alkyl ethers of diols, more preferably mono-C₁₋₄-alkyl ethers of diols having 2 to 12 carbon atoms, especially 2-methoxy-2-ethoxy-2-ethoxyethanol.

Examples of further suitable liquid media comprising a mixture of water and one or more organic solvents are described in U.S. Pat. No. 4,963,189, U.S. Pat. No. 4,703,113, U.S. Pat. No. 4,626,284 and EP-A-425,150.

When the liquid medium comprises organic solvent free from water, (i.e. less than 1% water by weight) the solvent preferably has a boiling point of from 30° to 200° C., more preferably of from 40° to 150° C., especially from 50 to 125° C. The organic solvent may be water-immiscible, water-miscible or a mixture of such solvents. Preferred water-miscible organic solvents are any of the hereinbefore-described water-miscible organic solvents and mixtures thereof. Preferred water-immiscible solvents include, for example, aliphatic hydrocarbons; esters, preferably ethyl acetate; chlorinated hydrocarbons, preferably CH₂Cl₂; and ethers, preferably diethyl ether; and mixtures thereof.

When the liquid medium comprises a water-immiscible organic solvent, preferably a polar solvent is included because this can enhance the solubility of the dye in the liquid medium. Examples of polar solvents include C₁₋₄-alcohols.

In view of the foregoing preferences it is especially preferred that where the liquid medium is organic solvent free from water it comprises a ketone (especially methyl ethyl ketone) and/or an alcohol (especially a C₁₋₄-alkanol, more especially ethanol or propanol).

The organic solvent free from water may be a single organic solvent or a mixture of two or more organic solvents. It is preferred that when the liquid medium is organic solvent free from water it is a mixture of 2 to 5 different organic solvents. This allows a liquid medium to be selected that gives good control over the drying characteristics and storage stability of the ink.

Liquid media comprising organic solvent free from water are particularly useful where fast drying times are required and particularly when printing onto hydrophobic and non-absorbent substrates, for example plastics, metal and glass.

The liquid media may of course contain additional components conventionally used in ink-jet printing inks, for example viscosity and surface tension modifiers, corrosion inhibitors, biocides, kogation reducing additives and surfactants which may be ionic or non-ionic.

Although not usually necessary, further colorants may be added to the ink to modify the shade and performance properties. Examples of such colorants include C.I. Direct Yellow 86, 132, 142 and 173; C.I. Direct Blue 307; C.I. Food Black 2; C.I. Direct Black 168 and 195; and C.I. Acid Yellow 23.

It is preferred that the composition according to the invention is ink suitable for use in an ink-jet printer. Ink suitable for use in an ink-jet printer is ink that is able to repeatedly fire through an ink-jet printing head without causing blockage of the fine nozzles.

Ink suitable for use in an ink-jet printer preferably has a viscosity of less than 20 cP, more preferably less than 10 cP, especially less than 5 cP, at 25° C.

Ink suitable for use in an ink-jet printer preferably contains less than 500 ppm, more preferably less than 250 ppm, especially less than 100 ppm, more especially less than 10 ppm in total of divalent and trivalent metal ions (other than any divalent and trivalent metal ions bound to a colorant of Formula (1) or any other colourant or additive incorporated in the ink).

Preferably ink suitable for use in an ink-jet printer has been filtered through a filter having a mean pore size below 10 μm, more preferably below 3 μm, especially below 2 μm, more especially below 1 μm. This filtration removes particulate matter that could otherwise block the fine nozzles found in many ink-jet printers.

Preferably ink suitable for use in an ink-jet printer contains less than 500 ppm, more preferably less than 250 ppm, especially less than 100 ppm, more especially less than 10 ppm in total of halide ions.

A fourth aspect of the invention provides a process for forming an image on a substrate comprising applying ink suitable for use in an ink-jet printer, according to the third aspect of the invention, thereto by means of an ink-jet printer.

The ink-jet printer preferably applies the ink to the substrate in the form of droplets that are ejected through a small orifice onto the substrate. Preferred ink-jet printers are piezoelectric ink-jet printers and thermal ink-jet printers. In thermal ink-jet printers, programmed pulses of heat are applied to the ink in a reservoir by means of a resistor adjacent to the orifice, thereby causing the ink to be ejected from the orifice in the form of small droplets directed towards the substrate during relative movement between the substrate and the orifice. In piezoelectric ink-jet printers the oscillation of a small crystal causes ejection of the ink from the orifice. Alternately the ink can be ejected by an electromechanical actuator connected to a moveable paddle or plunger, for example as described in International Patent Application WO00/48938 and International Patent Application WO00/55089.

The substrate is preferably paper, plastic, a textile, metal or glass, more preferably paper, an overhead projector slide or a textile material, especially paper.

Preferred papers are plain or treated papers which may have an acid, alkaline or neutral character. Glossy papers are especially preferred. Photographic quality papers are especially preferred.

A fifth aspect of the present invention provides a material preferably paper, plastic, a textile, metal or glass, more preferably paper, an overhead projector slide or a textile material, especially paper more especially plain, coated or treated papers printed with a compound as described in the first aspect of the invention, a metal chelate compound as described in the second aspect of the invention, a composition according to the third aspect of the invention or by means of a process according to the fourth aspect of the invention.

It is especially preferred that the printed material of the fifth aspect of the invention is a print on a photographic quality paper printed using a process according to the fourth aspect of the invention.

A sixth aspect of the present invention provides an ink-jet printer cartridge comprising a chamber and an ink suitable for use in an ink-jet printer wherein the ink is in the chamber and the ink is as described in the third aspect of the present invention. The cartridge may contain a high concentration ink and a low concentration ink, as described in the third aspect of the invention, in different chambers.

The invention is further illustrated by the following Examples in which all parts and percentages are by weight unless otherwise stated.

### EXAMPLE 1

### Preparation of

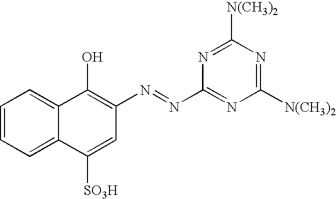

### Stage 1

### Preparation of

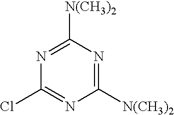

Dimethylamine (obtained from Aldrich, 40% in H₂O, 53 ml) was added drop-wise to a solution of cyanuric chloride (18.4 g) in acetone (200 ml) at 0 to 10° C. The resultant mixture was stirred for 2 hours at 0° C. and the overnight at room temperature. The next day the mixture was evaporated to give the product as 19.6 g of a white solid.

### Stage 2

### Preparation of

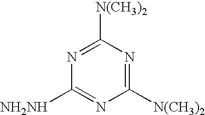

Hydrazine monohydrate (10 ml) was added drop-wise to a suspension of the chlorotriazine from stage 1 (10.1 g) in methanol. The reaction mixture was stirred overnight at room temperature, evaporated and the residue was slurried in ether (50 ml). The product was filtered off and pulled dry to give 9.2 g of a white solid.

### Stage 3

### Preparation of the Title Product

A solution of the hydrazine prepared in stage 2 (9.5 g) in water (500 ml) was added to a suspension of 4-sulfo-1,2-naphthoquinone (22.5 g, obtained from Acros) in cHCl (200 ml) at room temperature. The reaction mixture was then stirred for two hours at room temperature and the product was filtered off and then stirred in water (500 ml) at room temperature and pH 4 for 30 minutes. At the end of this time the product was pulled dry and stirred into acetone (500 ml) with ultra turrax and then filtered to give 28.2 g of a red solid.

### EXAMPLE 2

### Preparation of

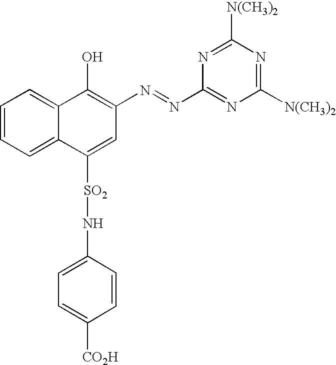

Phosphorous oxychloride (10 ml) was added to a suspension of the azo compound from Example 1 (6.8 g) in sulfolane (75 ml). The reaction mixture was stirred at 55-65° C. for 5 hours, added to a water/ice mixture (500 ml), filtered off and pulled dry. The resultant solid was dissolved in CHCl₃ (500 ml) separated from the small amount of residual water and dried with MgSO₄. This solution was then evaporated to give 7.8 g of a red powder. This powder was added, with stirring, to 4-aminobenzoic acid (3.2 g) in dimethylacetamide (200 ml). The reaction mixture was left stirring overnight at room temperature and the next day added to water (1000 ml), the crude product was collected by filtration and washed with water (200 ml). The solid was dissolved in water (500 ml) and the pH was adjusted to 10 with ammonium hydroxide solution. The pH of the solution was then adjusted to 3 by the addition of 2N hydrochloric acid, the resultant precipitate was collected by filtration, washed with water (200 ml) and dried to give 7.8 g of an orange solid.

### EXAMPLE 3

### Preparation of a 2:1 Nickel Complex of the Compound of Example 2

A solution of nickel acetate (1.06 g) in water (15 ml) was added dropwise to a suspension of the compound of Example 2 (2.93 g) in DMF (50 ml). The reaction mixture was stirred for 3 hours at 70° C. and then added to water (500 ml) and salted to 5% with NaCl. The product which formed was filtered off and washed with water (25 ml) at pH3. The product was then stirred in acetonitrile (50 ml) at reflux for 10 minutes, cooled and filtered to give 2.8 g of a dark green powder.

### EXAMPLE 4

### Preparation of

### Stage 1

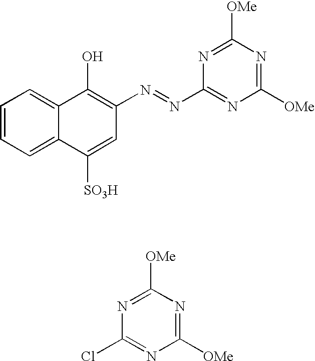

Purchased from Acros

### Stage 2

### Preparation of the Title Compound

Hydrazine hydrate (10 ml) was added to a suspension of the triazine formed in stage 1 (5 g) in methanol (50 ml) at 30° C. The reaction mixture was then stirred at room temperature for 1 hour and the hydrazino triazine which formed was filtered off as a white solid. The hydrazino triazine was then added to a solution of 4-sulfo-1,2-naphthoquinone (11.9 g) in water (200 ml) and cHCl (80 ml) and stirred at room temperature for 2 hours. The product was filtered off and pulled dry to give an orange solid.

### EXAMPLE 5

### Preparation of

### Stage 1

### Preparation of

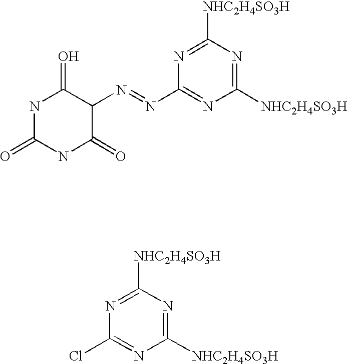

Prepared as in Example 1 Stage 1 except that taurine was used instead of dimethylamine.

### Stage 2

### Preparation of

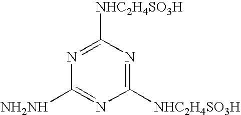

Prepared using the product of stage 1 following the procedure described in Example 1 stage 2.

### Stage 3

### Preparation of the Title Compound

The hydrazine prepared in stage 2 (3.6 g), alloxane (1.1 g) and cHCl (23 ml) were stirred at room temperature for 2 days. The reaction was then heated to 40° C. and stirring was continued for a further 16 hours. The product which formed was collected by filtration and was washed with water. The product was then dissolved in water (50 ml) adjusted to pH12 with 2N NaOH, dialysed, filtered and evaporated at 60° C.

### Preparation of Inks

Inks may be prepared from the compounds of Examples 1 to 7 by dissolving 3 g of a dye in 97 ml of a liquid medium consisting of 5 parts 2-pyrrolidone; 5 parts thiodiethylene glycol; 1 part Surfynol™ 465 and 89 parts water and adjusting the pH to between pH 8 to 9 with sodium hydroxide. Surfynol™ 465 is a surfactant from Air Products.

### Ink-Jet Printing

Inks prepared as described above may be filtered through a 0.45 micron nylon filter and then incorporated into empty print cartridges using a syringe.

These inks may then be printed onto either plain paper or a specialist ink-jet media using an ink-jet printer.

The prints so formed may be tested for ozone fastness by exposure to 1 ppm ozone at 40° C., 50% relative humidity for 24 hrs in a Hampden 903 Ozone cabinet. Fastness of the printed ink to ozone can be judged by the difference in the optical density before and after exposure to ozone.

Light-fastness of the printed image may be assessed by fading the printed image in an Atlas Ci5000 Weatherometer for 100 hours and then measuring the change in the optical density.

Optical density measurements may be performed using a Gretag spectrolino spectrophotometer set to the following parameters:

| | |
|---|---|
| Measuring Geometry | 45°/0° |
| Spectral Range | 380-730 nm |
| Spectral Interval | 10 nm |
| Illuminant | D65 |
| Observer | 2° (CIE 1931) |
| Density | Ansi A |
| External Filler | None |

### Further Inks

The inks described in Tables A and B may be prepared using the compounds and metal chelates of the various Examples as the dye component. Numbers quoted in the second column onwards refer to the number of parts of the relevant ingredient and all parts are by weight. The inks may be applied to paper by ink-jet printing.

The following abbreviations are used in Tables A and B:

PG=propylene glycol

DEG=diethylene glycol

NMP=N-methylpyrrolidone

DMK=dimethylketone

IPA=isopropanol

MeOH=methanol

2P=2-pyrrolidone

MIBK=methylisobutyl ketone

P12=propane-1,2-diol

BDL=butane-2,3-diol

CET=cetyl ammonium bromide

PHO=Na₂HPO₄ and

TBT=tertiary butanol

TDG=thiodiglycol

TABLE A

| Dye | | | | | | | Na | | | | |
|---|---|---|---|---|---|---|---|---|---|---|---|
| Content | Water | PG | DEG | NMP | DMK | NaOH | Stearate | IPA | MEOH | 2P | MIBK |
| 2.0 | 80 | 5 | | 6 | 4 | | | | | 5 | |
| 3.0 | 90 | | 5 | 5 | | 0.2 | | | | | |
| 10.0 | 85 | 3 | | 3 | 3 | | | | 5 | 1 | |
| 2.1 | 91 | | 8 | | | | | | | | 1 |
| 3.1 | 86 | 5 | | | | | 0.2 | 4 | | | 5 |
| 1.1 | 81 | | | 9 | | 0.5 | 0.5 | | | 9 | |
| 2.5 | 60 | 4 | 15 | 3 | 3 | | | 6 | 10 | 5 | 4 |
| 5 | 65 | | 20 | | | | | 10 | | | |
| 2.4 | 75 | 5 | 4 | | 5 | | | | 6 | | 5 |
| 4.1 | 80 | 3 | 5 | 2 | 10 | | 0.3 | | | | |
| 3.2 | 65 | | 5 | 4 | 6 | | | 5 | 4 | 6 | 5 |
| 5.1 | 96 | | | | | | | | 4 | | |
| 10.8 | 90 | 5 | | | | | | 5 | | | |
| 10.0 | 80 | 2 | 6 | 2 | 5 | | | 1 | | 4 | |
| 1.8 | 80 | | 5 | | | | | | | 15 | |
| 2.6 | 84 | | | 11 | | | | | | 5 | |
| 3.3 | 80 | 2 | | | 10 | | | | 2 | | 6 |
| 12.0 | 90 | | | | 7 | 0.3 | | 3 | | | |
| 5.4 | 69 | 2 | 20 | 2 | 1 | | | | | 3 | 3 |
| 6.0 | 91 | | | 4 | | | | | | 5 | |

TABLE B

| Dye Content | Water | PG | DEG | NMP | CET | TBT | TDG | BDL | PHO | 2P | PI2 |
|---|---|---|---|---|---|---|---|---|---|---|---|
| 3.0 | 80 | 15 | | | 0.2 | | | | | 5 | |
| 9.0 | 90 | | 5 | | | | | | 1.2 | | 5 |
| 1.5 | 85 | 5 | 5 | | 0.15 | 5.0 | 0.2 | | | | |
| 2.5 | 90 | | 6 | 4 | | | | | 0.12 | | |
| 3.1 | 82 | 4 | 8 | | 0.3 | | | | | | 6 |
| 0.9 | 85 | | 10 | | | | | 5 | 0.2 | | |
| 8.0 | 90 | | 5 | 5 | | | 0.3 | | | | |
| 4.0 | 70 | | 10 | 4 | | | | 1 | | 4 | 11 |
| 2.2 | 75 | 4 | 10 | 3 | | | | 2 | | 6 | |
| 10.0 | 91 | | | 6 | | | | | | 3 | |
| 9.0 | 76 | | 9 | 7 | | 3.0 | | | 0.95 | 5 | |
| 5.0 | 78 | 5 | 11 | | | | | | | 6 | |
| 5.4 | 86 | | | 7 | | | | | | 7 | |
| 2.1 | 70 | 5 | 5 | 5 | 0.1 | 0.2 | 0.1 | 5 | 0.1 | 5 | |
| 2.0 | 90 | | 10 | | | | | | | | |
| 2 | 88 | | | | | | 10 | | | | |
| 5 | 78 | | | 5 | | | 12 | | | 5 | |
| 8 | 70 | 2 | | 8 | | | 15 | | | 5 | |
| 10 | 80 | | | | | | 8 | | | 12 | |
| 10 | 80 | | 10 | | | | | | | | |

1. A compound of Formula (1) or a salt thereof: 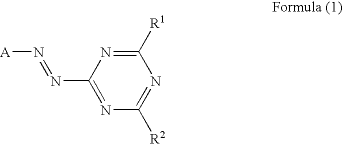
wherein:
A is optionally substituted aryl or optionally substituted heterocyclyl; and
R¹ and R² are independently H or a substituent.

2. A compound as claimed in claim 1 wherein at least one of A, R¹ or R² carries at least one acidic water solubilising group selected from the group consisting of —SO₃H, —CO₂H, —PO₃H₂ and —COSH.

3. A compound as claimed in claim 1 wherein A is optionally substituted phenyl, optionally substituted naphthyl, optionally substituted phenanthrenyl or optionally substituted 5- or 6-membered heterocyclyl.

4. A compound as claimed in claim 1 wherein A comprises a S, N or O either in the ring or as a substituent which is in such a position that it is, along with a N in the azo bond and a N in the triazinyl component able to ligate to a metal and so form a metal complex comprising a compound of Formula (1).

5. A compound as claimed in claim 1 wherein A carries at least one acidic water solubilising group selected from the group consisting of ˜O₃H, —CO₂H, —PO₃H₂ and —COSH.

6. A compound as claimed in claim 1 wherein A is 1-hydroxy-2-naphthyl or an analogue carrying at least one acidic water solubilising group selected from the group consisting of ˜O₃H, —CO₂H, —PO₃H₂ and —COSH.

7. A compound as claimed in claim 1 wherein R¹ and R² are independently selected from; H; —NR^{a}R^{b}, wherein R^{a} and R^{b} are each independently H or optionally substituted alkyl, optionally substituted C₁₋₄-alkoxy, SH or SR^{c}, wherein R^{c} is optionally substituted alkyl.

8. A compound as claimed in claim 1 which is free from fibre reactive groups.

9. A metal chelate compound comprising a compound of Formula (1) as described in claim 1.

10. A metal chelate compound as claimed in to claim 9 wherein the metal is nickel or copper.

11. A composition comprising a compound of Formula (1) as described in claim 1 or a metal chelate compound comprising a compound of Formula (1) as described in either claim 9 or claim 10, or a mixture thereof, and a liquid medium.

12. A composition as claimed in claim 11 which is ink suitable for use in an ink-jet printer.

13. A process for forming an image on a substrate comprising applying ink suitable for use in an ink-jet printer, as described in claim 12, thereto by means of an ink-jet printer.

14. A material printed by means of a process according to the claim 13.

15. A material as claimed in claim 14 which is a print on a photographic quality paper.

16. An ink-jet printer cartridge comprising a chamber and an ink suitable for use in an ink-jet printer wherein the ink is in the chamber and the ink is as described in claim 12.
